(19) United States
(12) Patent Application Publication
(10) Pub. No.: US 2014/0135891 A1
(43) Pub. Date: May 15, 2014

(54) Balloon Catheter for Curved Vessels

(71) Applicant: BIOTRONIK AG, Buelach (CH)

(72) Inventors: Stefanie Poehlmann, Rostock (DE); Alexandre Amido, Rostock (DE); Matthias Wesselmann, Ruedlingen (CH)

(73) Assignee: BIOTRONIK AG, Buelach (CH)

(21) Appl. No.: 14/071,612

(22) Filed: Nov. 4, 2013

Related U.S. Application Data
(60) Provisional application No. 61/725,492, filed on Nov. 13, 2012.

(51) Int. Cl. A61F 2/958

(52) U.S. Cl. CPC A61F 2/958
USPC 623/1.11

(57) ABSTRACT

A balloon catheter for the treatment of a stenosis in a bodily vessel (2), in particular for introducing a stent (1) into the bodily vessel (2), wherein the balloon (10) is formed in such a way that, in the expanded state, it adopts the curved or bent three-dimensional form of the bodily vessel (2).

### CROSS REFERENCE TO RELATED APPLICATIONS

This application claims benefit of priority to U.S. provisional patent application Ser. No. 61/725,492 filed Nov. 13, 2012; the content of which is herein incorporated by reference in its entirety.

### TECHNICAL FIELD

The present invention relates to a balloon catheter, including an expandable balloon for the treatment of stenoses in bodily vessels. The invention further relates to a system for introducing a stent into a bodily vessel.

### BACKGROUND

A stenosis of a bodily vessel is understood to mean the constriction of the vessel diameter. Constrictions may occur in bodily vessels, in particular in arteries, veins or similar hollow organs, as a result of deposits. The flow through these vessels is thus restricted and, in the worst case, inhibited. Bodily liquids no longer pass through the bodily vessels, and regions located downstream are no longer supplied or are not supplied sufficiently.

Stenoses of this type in arteries or veins can be treated medically by a method known as angioplasty. In the case of an angioplasty, the constricted vessels are widened again mechanically, whereby the diameter of the bodily vessels through which liquid can flow is increased again. During the process, a balloon catheter comprising an expandable balloon is guided into the bodily vessel in such a way that the expandable balloon is located at the point of the stenosis, that is to say the constriction. The bodily vessel is widened again as a result of the expansion of the balloon. The vessel walls are expanded and the deposit is pressed against the vessel wall.

To ensure permanent widening, a vessel support or what is known as a “stent” is often introduced into the bodily vessel at the point in question. In this case, the balloon and/or stent often also have a coating, which delivers medically effective substances over a predefined period so as to improve the healing process and/or to prevent the formation of a renewed constriction (restenosis).

Within the scope of this application, the treatment of a stenosis in a bodily vessel is therefore understood to mean the treatment of a constriction in a bodily vessel, in particular in an artery or vein, wherein a balloon catheter is introduced into the bodily vessel in question. The treatment comprises the widening of the constriction by means of an expandable balloon of the balloon catheter, either with or without introduction of a stent, either with or without coating of the balloon and/or stent.

Within the scope of the application, a stent is understood to mean a metal or polymer braid, which can be introduced into the bodily vessel. In doing so the stent supports the bodily vessel, at least over a predefined period of time. Within the scope of this application, the stent may be both permanently and biologically decomposable or degradable.

Various balloon catheters comprising an expandable balloon are known in the prior art. Within the scope of this application, the terms “expansion/expand”, “dilation/dilate” and “distension/distend” are considered to be synonymous, wherein the term “expansion/expand” is used within the scope of this application.

The balloon catheters known in the prior art all have the following key components: a balloon, a tip, an inner lumen for a guide wire, and a lumen for acting on the balloon by means of a fluid as well as the appropriate possibilities for connection to a pressure source for the fluid.

Within the scope of this application, a lumen is understood to mean the free cross section of a hollow body through which fluid can flow. In the simplest case of a cylindrical tube, the lumen is produced accordingly as an inner cross section of the tube.

A balloon catheter has a shaft, in the distal region of which the expandable balloon is located. The connection possibilities (usually one or more Luer taper connections) for the supply of the guide wire or for the fluid provided to act on the balloon are located in the proximal region or at the proximal end of the balloon catheter.

Within the scope of this application, the proximal end of a balloon catheter is understood to mean the end located in the hand of the handler, outside the body/bodily vessel. The distal end of the balloon catheter is accordingly the tip of the balloon catheter guided into the bodily vessel. The positional indications “proximal” (closer to the handler) and “distal” (closer to the tip of the balloon catheter located in the bodily vessel) are to be understood accordingly.

A guide wire, which is equipped with X-ray markers or is completely visible for X-ray radiation, is generally placed in the bodily vessel beforehand. The balloon catheter is inserted via this guide wire, with the tip at the leading end. The tip of the balloon catheter is very flexible so as to prevent damage to the walls of the bodily vessel and is coated in such a way that it can slide in the bodily vessel with minimal friction. In addition, it is visible under X-ray radiation. The guide wire is guided in the inner lumen of the balloon catheter.

The balloon catheter is placed with the aid of the guide wire such that the balloon is at the point of the stenosis in the vessel. So as to ensure reliable placement, the shaft and/or the balloon has corresponding X-ray markings. A fluid then acts on the balloon via the corresponding lumen and the balloon is expanded. The deposits are pressed against the vessel wall during this process and the vessel is widened. During this process a pressure of 6 bar (4 to 8 bar, low-pressure expansion) or, in the case of high-pressure expansion, a pressure of more than 16 to 18 bar or up to 40 bar is applied to the balloon.

Depending on the arrangement of the lumen of the balloon catheter, a distinction is made in the prior art between an over-the-wire system, a rapid-exchange system or a fixed-wire system. In an over-the-wire system, the inner lumen for the guide wire and the lumen for acting on the balloon by means of a fluid are arranged coaxially. The guide wire is located in the inner shaft comprising the inner lumen, and the lumen for acting on the balloon is formed by the outer shaft having a larger inner and outer diameter. Both lumens extend from the proximal end to the distal end of the balloon catheter. In a rapid-exchange system (sometimes also known as a monorail system or single-operator system), the lumen for the guide wire does not continue as far as the proximal end of the balloon catheter, but is only provided in the distal portion of the balloon catheter (approximately 20 cm). This allows a quick replacement of the balloon catheter and the use of shorter guide wires. The lumen for the guide wire and the lumen for acting on the balloon by means of a fluid may be arranged coaxially or in parallel. Fixed-wire balloon catheters have a guide wire that is positioned fixedly in its lumen.

Within the scope of this application, the lumen for the guide wire is always referred to as an inner lumen (and inner shaft accordingly) and the lumen for acting on the balloon by means of a fluid is always referred to as an outer lumen, irrespective of a coaxial or parallel arrangement. The interior of the balloon, which can be filled with fluid and pressurized, is referred to as a balloon lumen or balloon interior within the scope of this application.

If balloon catheters of this type according to the prior art are used to implant stents in bodily vessels, a stent is applied to the expandable balloon. This is also referred to in the prior art as crimping. As the balloon expands, the stent is then distended and pressed against the vessel wall. In this case, the stent is made of a material that, once expanded, retains the form obtained from the expansion process.

Different balloons and stents are known in the prior art in a wide range of sizes. For example, a balloon that has a diameter of 3.0 mm in the expanded state and a length of 20 mm for coronary use is to be understood as a standard size in the prior art. The respective stent has similar dimensions in its expanded state.

The unexpanded balloon is folded over the inner shaft, possibly with the stent pressed thereagainst, such that a minimal diameter is produced in the unexpanded state so as to guide the balloon catheter as easily and as smoothly as possible into the bodily vessel at the point of the stenosis. Upon expansion, both the balloon and, where applicable, the stent fastened thereon adopt a substantially cylindrical form.

This leads to problems with curved bodily vessels, in particular if the stenosis occurs at a curvature of the bodily vessel. In this case, the expansion of the balloon leads to an undesirable and unphysiological straightening of the bodily vessel, caused by the high flexural rigidity of the inflated balloon, which is maintained in particular with use of a permanent stent. If a stent is introduced into a vessel curved in this manner, the part of the vessel that is supported directly by the stent is straightened. The adjacent vessel portions retain their previous form substantially, such that the bodily vessel is kinked at the edges of the stent, as illustrated in FIG. 1.

FIG. 1 shows a stent 1 in a bodily vessel 2 after implantation with a balloon catheter according to the prior art described above. Due to the unphysiological straightening in the region of the implanted stent 1, the vessel 2 is kinked at the edge of the stent 1. In this case, the stent 1 presses into the vessel wall in the outer region 1a of the kinked vessel 2, whereby high stresses are introduced into the bodily vessel 2 in this area. According to recent investigations, this leads to an increase in the risk of restenosis. The stent 1 does not lie well against the wall of the vessel 2 in the inner region 1i of the kinked vessel. A region having a considerably reduced flow is thus formed at this point, whereby the risk of restenosis likewise increases.

The object of the present invention is therefore to design a balloon catheter of the type described above, in particular a balloon catheter for introducing a stent into a bodily vessel, in such a way that the balloon in the expanded state emulates the natural progression of the bodily vessel as closely as possible. A straightening of the bodily vessel as a result of the expanded balloon, in particular as a result of a stent, which is pressed against the vessel wall by means of the expanded balloon, is to be avoided in the present invention.

In accordance with the invention, the balloon is formed in such a way that, in the expanded state, it adopts a form that deviates from the form of a cylinder, and in particular the balloon has a bent or wound three-dimensional form in the expanded state. The curved vessel is primarily straightened by the substantially cylindrical form of the expanded balloon of a balloon catheter according to the prior art. This is avoided by the present invention. The form of the expanded balloon in a balloon catheter according to the invention is not cylindrical. The straightening and the associated negative effects as a result of the expansion of the balloon, and the optionally associated implantation of the stent, are thus avoided.

Within the scope of the application, the expanded state of the balloon is understood to mean the fact that the balloon, in its intended position in the bodily vessel, has been acted on by a fluid and has been expanded. This includes both the above-described low-pressure expansion and the high-pressure expansion.

The balloon is advantageously shaped in such a way that, in the expanded state, it is adapted to the form of the surrounding bodily vessel, and in particular has a bent or wound three-dimensional form. The above-described problems occur in the case of bodily vessels of which the form deviates from a straight cylindrical form, that is to say the stenosis has formed at a point where the bodily vessel is bent. Due to the balloon catheter according to the invention, the balloon can advantageously be expanded at this point and the bodily vessel can thus be distended, without the bodily vessel being straightened. In its expanded state, the balloon has a bent or wound three-dimensional form, which corresponds to the form of the bodily vessel to be treated. When a stent is introduced into the bodily vessel, said stent is thus also advantageously distended to a bent or wound three-dimensional form. The distended form of the stent corresponds to the form of the bodily vessel, such that the stent is adapted optimally to the bodily vessel and the bodily vessel is not straightened by the introduced stent, but remains in its natural three-dimensional form. The above-described effects of the prior art at the inner radius (flow dead points) and at the outer radius (high stresses in the vessel wall as a result of the stent) are thus avoided and the risk of restenosis is lowered on the whole.

In accordance with an advantageous variant of the invention, the balloon is composed from at least two, preferably three, individual segments, wherein at least one segment has an area that is not rectangular. In accordance with this variant of the invention, the balloon is composed from a plurality of, at least two, preferably three, individual segments, wherein the area of at least one segment deviates from a rectangular area. The balloon can thus be composed in such a way that, in the expanded state, a non-cylindrical, bent or wound form is produced.

Within the scope of the invention, a segment is understood to mean a piece of a balloon that, together with at least two other segments, forms the complete balloon. A segment is thus an individual piece of the surface of the balloon of any area. A sphere, such as a football, can be composed from a multiplicity of hexagons and pentagons. Within the scope of this application, these hexagons and pentagons would be understood as segments having a hexagonal and pentagonal area respectively, and not a rectangular area. The respective contact regions of the balloon with the shaft of the balloon catheter are not understood to be segments within the scope of the application. The contact regions or contact points instead form a spherical segment, which cannot be defined exactly, and are therefore not considered to be a segment within the scope of this application and within the meaning of this variant of the invention. Within the scope of this variant, the main part of the balloon, that is to say the primary form of the balloon without the contact region of the balloon with the shaft of the balloon catheter, is composed from at least two, preferably three, individual segments.

Within the scope of the invention, a segment is not a notional division of the balloon into individual portions. A balloon catheter of this variant of the invention has a balloon, which is composed from a plurality of segments or individual parts. The individual segments are interconnected in such a way that the balloon produced, in its intended state, can be acted on by a fluid in a pressure-tight manner.

In this variant of the invention, at least one segment of the balloon has an area that is not rectangular. A segment having a rectangular base area corresponds to a cylinder sleeve and would accordingly produce a cylindrical form when composed three-dimensionally. Due to the deviation from the form of a rectangle, a three-dimensional form is achieved that differs from the form of a cylinder. The bending of the balloon is set by the orientation of the segment edges.

The individual segments are preferably welded or glued together at their bordering edges, wherein the bordering edges overlap at least slightly, so as to ensure a welded or glued bond. The entire balloon is formed by welding or gluing the individual segments. The individual segments are thus connected in a pressure-resistant manner to form an expandable balloon.

A balloon catheter of this variant of the invention can therefore be produced by cutting individual segments from balloon material. The individual segments are then welded or glued together in such a way that the desired three-dimensional form of the balloon is produced. The connection regions between the balloon and the shaft of the balloon catheter can be formed in different ways in this variant of the invention.

On the one hand, balloon material can be heated and stretched under pressure, as in the prior art. This leads in the prior art to a cylindrical balloon. The edge regions of said balloon are cropped and are glued or welded to the segments of this embodiment of the invention. The edge regions are then used for connection of a balloon of this embodiment of the invention to the shaft of the balloon catheter. Alternatively, specific edge regions can also be cut from balloon material and connected to the segments of this embodiment of the invention by means of welding or gluing.

In the expanded state, the balloon advantageously has a form that consists of at least two cylindrical portions, wherein the main axes of at least two cylindrical portions have an angle of more than 0° and less than 180°, preferably more than 0° and less than 90°.

In this variant of the invention, the main axis of a cylindrical portion is understood to mean the axial axis of symmetry of the cylindrical portion, that is to say the axis from the midpoint of the hypothetical circular base area to the midpoint of the hypothetical circular top area of the cylindrical portion.

This is the simplest and most expedient implementation of this embodiment of the invention. The balloon expediently consists of three different segments. Two segments of rectangular area are connected via a third segment to an area that is not rectangular. The two segments of rectangular area form cylindrical portions. The rectangular area of the segments is the lateral surface of the cylinder of this portion. The edges of these two segments are connected to the edges of the third segment, which has an area that is not rectangular. As a result, the main axes of the two cylindrical portions thus have an angle that is greater than 0° and less than 180°. That is to say, in the expanded state of the balloon of this variant, two cylinders are connected via a segment that is not cylindrical, whereby a curvature is automatically produced. The two main axes of the cylindrical portions are no longer arranged in a line, and the balloon as a whole is curved or bent in the expanded state.

The curvature of this variant of the invention corresponds to the curvature and the natural state of the bodily vessel into which the balloon is introduced for expansion and in particular for introduction of a stent. The curvature of the expanded balloon can advantageously be varied as desired, similarly to the angle between the cylindrical segments of the balloon, wherein the handler chooses between different balloons of this type in accordance with the bodily vessel to be treated.

Depending on the cut of the third segment between the cylindrical segments, the angle between the main axes of the cylindrical segments (and thus the curvature of the expanded balloon) can be made larger or smaller. The radius of the curvature can be controlled in this variant of the invention by the size, number and angle of the individual segments relative to one another. If more than one third segment is inserted between the two cylindrical segments, a bend can be defined more finely/precisely. The more and finer segmented, the less sharply is the balloon bent in the expanded state. In addition, any desired three-dimensional form of the balloon can advantageously be achieved in the expanded state via the number and cut of the segments.

The individual segments are expediently not only connected to one another, but also to themselves, in such a way that a hollow body is produced. A segment is preferably connected to itself in such a way to form a hollow body that the connection lies in line with the connection of the adjacent segment to itself. This connection line is preferably the inner radius of the curved balloon in the expanded state.

This variant of the invention thus defines a balloon catheter comprising a balloon, which in the expanded state adopts any wound or bent three-dimensional form. When treating a stenosis in a bodily vessel, in particular when introducing a stent into this bodily vessel, a balloon catheter according to this variant of the invention is advantageously selected in such a way that the curved or bent three-dimensional form of the balloon in the expanded state matches the curved or bent three-dimensional form of the bodily vessel. In this case, the handler selects the balloon catheter of this variant of the invention accordingly from a plurality of such variants of the inventions with balloons having a wide range of wound or bent forms and sizes. The straightening of the bodily vessel and the associated problems of the prior art are avoided in this variant of the invention.

In another variant of the invention, at least one wire is suitably connected to the material of the balloon in such a way that the resilience of the balloon in the unexpanded state differs in at least one portion along a first curve over the balloon from the resilience along a second curve over the balloon.

Within the scope of this application and this variant of the invention, the resilience of the balloon is understood to mean the response of the balloon in the event of a pressure change/pressure increase. The balloon is pressurized by means of a fluid in the balloon interior and is thus expanded. The resilience defines the strength of the expansion at a predefined pressure. A balloon of higher resilience expands more severely than a balloon of lower resilience under identical pressure. In the English literature, resilience is also referred to as compliance. The level of resilience of a balloon in the radial direction has little direct influence on how well a balloon nestles against the vessel. This is more dependent on the flexural rigidity in the expanded state.

Within the scope of the invention, a curve is understood to mean a continuous sequence of points in geometric space. In the simplest case, a curve is a straight line over the balloon material.

In this variant of the invention, a non-cylindrical form of the balloon of the balloon catheter according to the invention is achieved by a selective, local change to the resilience of the balloon in a predefined portion along a predefined line. Due to the connection of a wire to the balloon material, a selective modification to the resilience of the balloon can be made. In the simplest case, the resilience of the balloon is reduced along the connection line between the balloon and wire.

A wire is preferably welded or glued along a curve to the material of the balloon, preferably in the interior of the balloon, or is integrated into the balloon material.

This variant of the invention comprises a wide range of embodiments. The wire may expediently have the same length as the balloon from the proximal end to the distal end. Alternatively, both shorter and longer wires are also possible. Different forms of wires (round wires, profiled wires or flat strips as well as straight wires, spiral wires or the like) and materials (metal or non-metal spring steel, fishing line) are also included in this variant of the invention. In this case, the choice of form, material and length of the wire depends on the desired form of the balloon in the expanded state and on the preferred fabrication. Within the scope of this application, the term “wire” is therefore not limited to metal wires, but generally describes a wire-like line.

In this variant of the invention, a balloon having a strong curvature as a result of the connection to a short, very rigid wire can thus be achieved. Due to a close linking of a wire of this type to the balloon material, for example by welding, the resilience of the balloon along the wire is reduced drastically. The balloon thus stretches to a much lesser extent along the wire in the expanded state, whereby a curved three-dimensional form of the balloon is achieved. Due to the use of a plurality of different wires of this type, any three-dimensional form of the balloon in the expanded state can expediently be achieved. Similarly to the first variant of the invention, a balloon catheter comprising a balloon of predefined three-dimensional form in the expanded state can thus be selected by the handler in such a way that the natural form of the bodily vessel is emulated. Correspondingly, the bodily vessel is not straightened when the balloon is expanded, in particular when a stent is introduced into the bodily vessel, whereby the described disadvantages of the prior art are avoided.

In a preferred embodiment of this variant, the curve in the unexpanded state of the balloon is a straight line that preferably extends from the proximal end to the distal end of the balloon.

In this embodiment of the invention, a straight wire is connected to the balloon over the entire length of the balloon from the proximal end to the distal end. The wire is preferably welded or glued to the balloon material at a number of points in the balloon interior, preferably along the entire length. In this variant of the invention, the rigidity of the balloon along a straight line from the proximal end to the distal end is increased. The resilience of the rest of the balloon remains uninfluenced and the balloon is merely more rigid along the wire from the proximal end to the distal end. Accordingly, the balloon expands to a much lesser extent along this line in the event of expansion as a result of the application of force by means of a fluid. In the expanded state, the balloon thus adopts a bent, three-dimensional form, similar to a banana. In this case, the wire can be tacked or completely welded or glued at points in the balloon interior. Alternatively, the wire can be integrated directly into the balloon material. An embodiment of this type can be produced, for example, by placing and fixing a wire on the inner balloon and by inserting this construction into an outer balloon having a proximal neck of corresponding size.

In a particularly preferred embodiment of this variant, the straight wire (or inner shaft of particularly high tensile strength) is only linked centrally to the balloon material, for example at the proximal and distal balloon neck. In this variant of the invention, the axial stress in the balloon membrane generated by the internal pressure in the balloon is absorbed partially or completely by the wire. The balloon membrane therefore is not tensioned over the entire circumference in the axial direction. A curved three-dimensional form similar to a banana is thus produced automatically. The balloon membrane is tensioned over the outer face of the curvature, and lies in folds over the inner face. This particularly preferred form adapts to the three-dimensional winding of the bodily vessel and is thus “comfortable”.

The resilience of the balloon along the inner face of the curvature is accordingly reduced automatically and the balloon, in the expanded state, follows the natural curvature of the bodily vessel. The described disadvantages of the prior art are thus avoided in an optimal manner.

In another preferred embodiment of this variant, the curve is a helix, wherein, along a first straight axis over the balloon from the proximal end to the distal end, less balloon material is located between two adjacent points of intersection of the first curve and the helix than between the adjacent points of intersection of a second curve over the balloon from the proximal end to the distal end and the helix, wherein the first and second curve are straight lines in the unexpanded state of the balloon.

In a particularly preferred form of this variant, the helix and the first and second axis extend from the proximal end to the distal end of the balloon, wherein the first and second axis are preferably located on opposite sides of the balloon. In this embodiment of the inventions, the windings of a helix are connected to the balloon material, wherein the helix, in the simplest case, has the same length as the balloon. In this case, the helix windings are connected to the balloon material along two straight lines. These two straight lines are preferably arranged opposite one another, wherein more balloon material is located along one straight line between two adjacent helix windings than along the opposite straight line. In other words, the balloon material along one of the straight lines is nested between two adjacent links similarly to a curtain, that is to say the length of the balloon material between two adjacent windings of the helical wire is greater than the distance between the two windings. Accordingly, more balloon material is thus located between the adjacent helix windings than along the opposite straight line. If the balloon is expanded by application of the fluid, the balloon can stretch to a greater extent along the straight line having a material excess than along the opposite straight line. The opposite straight line thus forms a curve of greater rigidity, whereby a curved three-dimensional form of the balloon is produced similarly to the previous embodiment.

The variants described in the previous sections apply analogously for the connection of the helical wire to the balloon material and for the materials.

In this variant of the invention, any curved three-dimensional forms of the balloon can be produced in the expanded state, depending on how the first and second curves are selected. Both can extend over the entire length of the balloon from the proximal end to the distal end or less. In some applications, it is also expedient if both curves cross, whereas in other applications they can also be arranged in a line. Due to the reduction in the resilience of the balloon along any point of the first curve due to the reduction in the balloon material between the helix windings, a curvature is always achieved at these points. Accordingly, variants similar to the previous section having more than two curves are also possible. The balloon may also expediently have portions that are provided differently with curves and different helical wires.

In a further variant of the invention, at least two wires are located inside the balloon and are interconnected in such a way that the resilience of the balloon in the unexpanded state differs in at least one portion along a first curve over the balloon from the resilience along a second curve over the balloon. In this case, a helical wire is preferably connected to a first wire at least at two adjacent windings of the helix, wherein both wires preferably extend from the proximal end to the distal end of the balloon. The first wire is particularly preferably straight.

In a similar variant of the invention, a helical wire is connected to a first wire at least at two adjacent windings of the helix and is connected to a second wire at least at two adjacent windings of the helix. The length of the first wire between two adjacent windings of the helix preferably differs from the length of the second wire between two adjacent windings of the helix, wherein both the helical wire and the first and second wire preferably extend from the proximal end to the distal end of the balloon. The first and the second wire are particularly preferably straight, and the first wire is in particular connected to the helical wire on the side of the helix opposite the second wire. Both the helical wire and the first and second wire expediently extend over the entire length of the balloon from the proximal end to the distal end.

The last two variants described resemble the variant of the directly preceding paragraphs. In these variants, a helical wire is introduced into the balloon interior as a sort of frame. The resilience of the balloon is manipulated deliberately and locally by the introduction of one or more wires connected to the windings of the helical wire. The spacing or possible distancing can thus be manipulated in accordance with the applied pressure of the helix windings of the wire and thus of the balloon. In these variants, the resilience is therefore analogously changed locally, and curved or bent three-dimensional forms of the balloon of the balloon catheter in the expanded state are achieved. In these variants too of the balloon catheter according to the invention, the straightening of the bodily vessel, in particular when introducing a stent into the bodily vessel, and the associated disadvantages of the prior art can therefore be avoided.

In a further alternative variant of the balloon catheter according to the invention, it has in the distal region an inner shaft with an inner lumen and an outer second lumen, wherein the second lumen has a fluidic connection to the balloon interior in such a way that the balloon can be expanded by a fluid in the second lumen and the length of the balloon from the proximal end to the distal end is greater than the length of the inner shaft from the proximal end of the balloon to the distal end of the balloon.

The length of the balloon of diameter d from the proximal end to the distal end L*(1−r/(r+d)) is preferably greater than the length L of the inner shaft from the proximal end of the balloon to the distal end of the balloon so as to follow a radius of curvature r of the bodily vessel.

In this variant of the invention, the balloon has a greater length from the proximal end to the distal end than the inner shaft. This leads to a non-cylindrical form in the expanded state of the balloon. The balloon according to this variant of the invention adapts to the wound or bent three-dimensional course of the bodily vessel in which the balloon is expanded. This is ensured by the material excess of the balloon as a result of its greater length. The balloon is longer or more balloon material is provided than is actually necessary. The shorter inner shaft follows the course of the bodily vessel over the shortest path, that is to say it practically cuts the curvatures and bends of the course of the vessel. Due to the greater length, the balloon can follow the natural bent or wound three-dimensional course of the bodily vessel however. A forced straightening of the vessel as with a matching length of the inner shaft and expanded balloon according to the prior art with the above-described disadvantages is thus avoided.

The balloon catheter preferably has means, with which the size of the difference in length between the balloon and the inner shaft can be set variably. In particular, the distal end of the balloon is pressure tight and movable over the inner shaft. A means of this type would be a sliding seal for example.

With the balloon catheter according to the invention, a stent in particular is applied to the balloon.

The individual variants of the invention can be combined with one another as desired.

The application further relates to a system for introducing a stent into a bodily vessel, comprising a balloon catheter as described in the paragraphs above.

With the aid of the balloon catheter according to the invention and the system according to the invention for introducing a stent into a bodily vessel, the straightening of the bodily vessel as a result of the expansion of the balloon and the introduction of the stent is avoided. The natural flow conditions of the bodily vessel before the stenosis are thus reproduced/obtained in a much improved manner. At the same time, the disadvantages described in the prior art (flow dead points in the inner region with an inserted stent; pressing of the stent into the vessel wall in the outer region of the curved bodily vessel) are avoided and the risk of a restenosis is lowered accordingly.

### DESCRIPTION OF THE DRAWINGS

The invention will be explained in greater detail hereinafter on the basis of the exemplary embodiments illustrated in the figures, in which:

FIG. 1 shows a bodily vessel with a stent after insertion of the stent with a balloon catheter according to the prior art,

FIG. 2 shows a variant of the balloon catheter according to the invention with a balloon formed from individual segments,

FIG. 3 shows the three-dimensional illustration of the balloon of the variant with three segments,

FIG. 4 shows a variant of the balloon catheter according to the invention with a balloon with a wire,

FIG. 5 shows a variant of the balloon catheter according to the invention with a balloon with a helically wound wire and a first straight wire and a second straight wire, and

FIG. 6 shows a variant of the balloon catheter according to the invention with a balloon that has a greater length from the proximal end to the distal end than the inner shaft.

### DETAILED DESCRIPTION

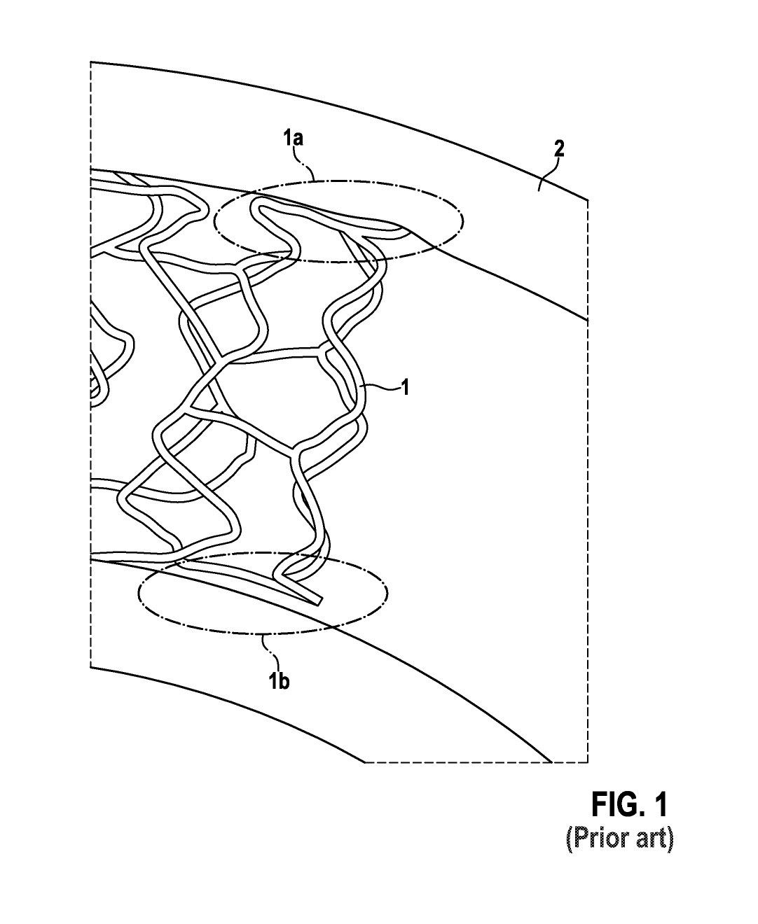

FIG. 1 has already been described during the discussion of the prior art.

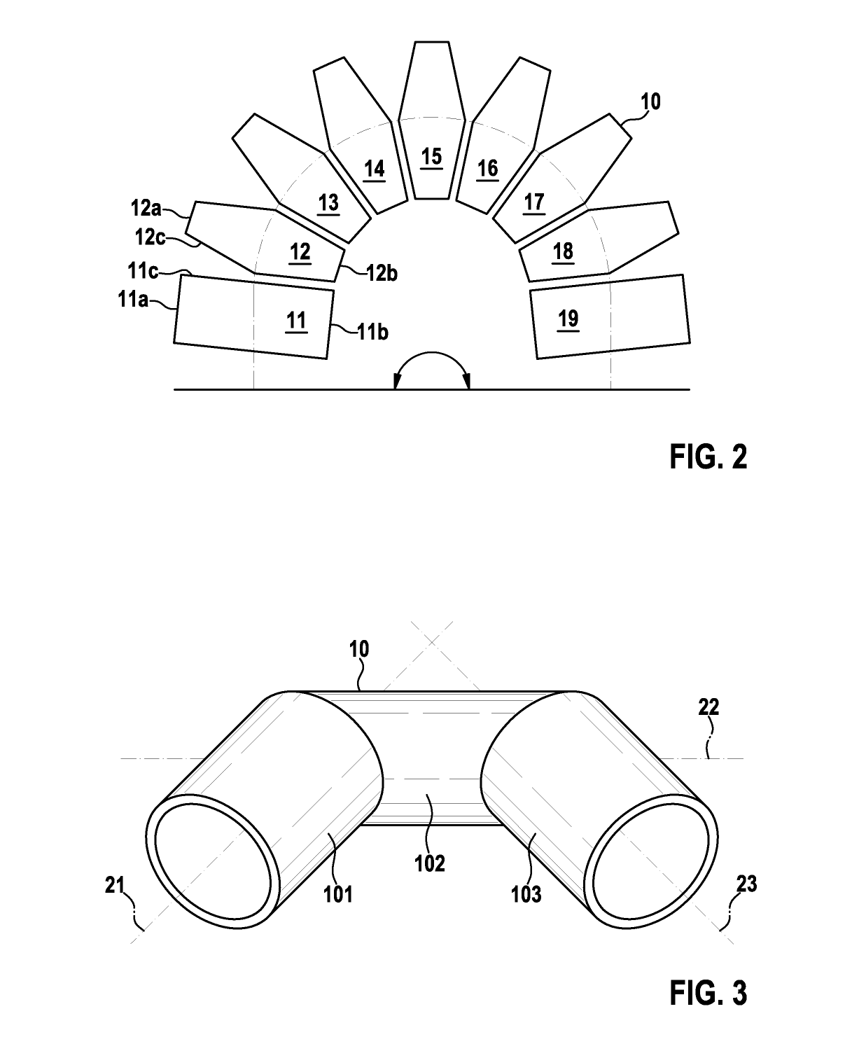

FIG. 2 shows a schematic view of a balloon 10 that consists of the individual segments 11 to 19, wherein the areas of the individual segments are illustrated. In the embodiment illustrated in FIG. 2, the balloon 10 consists of nine individual parts 11 to 19. In this case, only the segments 11 and 19 have a rectangular area. The other segments 12 to 18 have an area that is not rectangular and is in the form of an elongate hexagon. The actual balloon 10 is composed from the individual segments 11 to 19, which will be described by way of example with reference to the segments 11 and 12.

In segment 11, the two side edges 11a and 11b are interconnected. A cylindrical hollow body is thus produced. In segment 12, the two side edges 12a and 12b are likewise interconnected so that a hollow body is likewise produced. The two segments 11 and 12 are then interconnected at their side edges 11c and 12c. Due to the fact that the area of segment 12 is not rectangular, the form of the combined segments 11 and 12 deviates from the cylinder form. A hollow body having a bend is produced. All segments 11 to 19 are interconnected analogously. A balloon 10 can thus be formed from the individual segments 11 to 19, such that the balloon 10, in its expanded state, has a bent three-dimensional form in the form of a U. As indicated, the radius of the bend can be predefined by the number and dimension of the individual segments 12 to 18.

FIG. 3 shows a schematic three-dimensional illustration of a variant of a balloon catheter according to the invention with the simplest form of a balloon 10 formed from individual segments. The balloon 10 consists of three segments 101, 102 and 103 and is illustrated in its expanded state. The balloon 10 consists of two segments 101 and 103 having a rectangular area and one segment 102 having an area that is not rectangular. Due to the area that is not rectangular of the middle segment 102, a bent three-dimensional form of the balloon 10 is reached in its expanded state. The main axes 21, 22 and 23 of the three cylindrical portions 101, 102 and 103 each have an angle of more than 0° and less than 90° to one another.

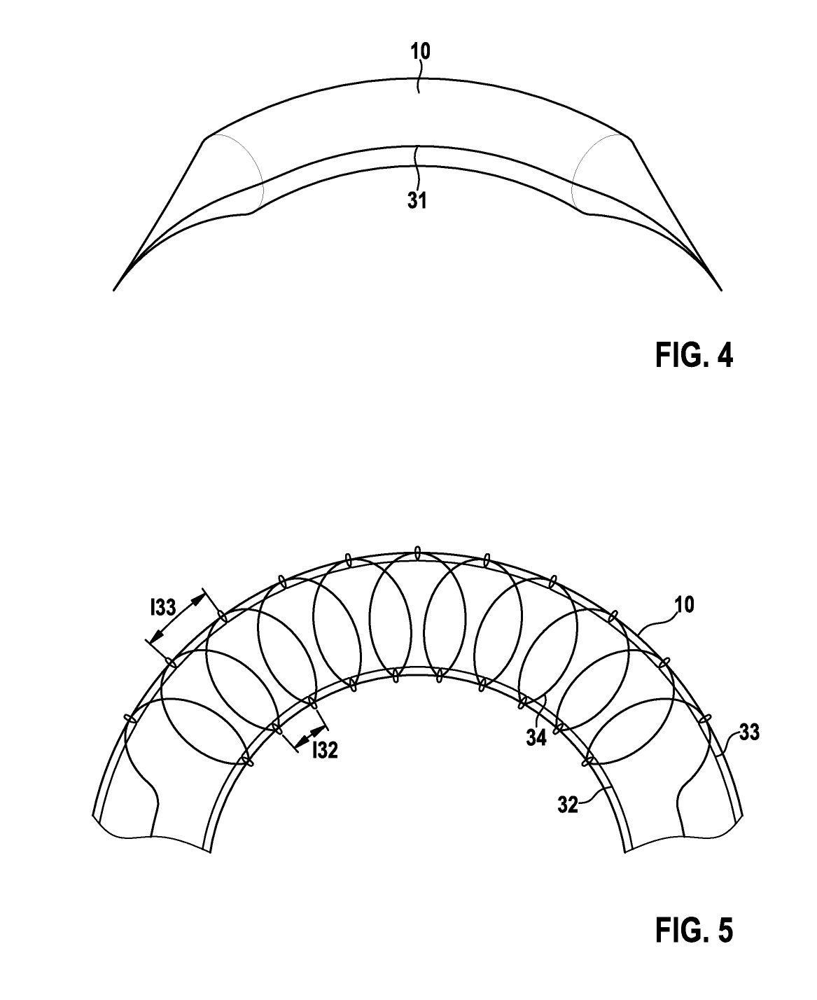

FIG. 4 shows a schematic view of the balloon 10 of a variant of a balloon catheter according to the invention, wherein the balloon 10 has a wire 31. The wire 31 extends in this variant of the invention as a straight line over the entire length of the balloon 10 from the proximal end to the distal end. The wire 31 is connected to the balloon material of the balloon 10 (welded, glued or introduced into the balloon material). As a result of the wire 31, the rigidity of the balloon 10 is changed locally. More specifically, the rigidity along the wire 31 is increased sharply. The balloon 10 can thus stretch to a much lesser extent along the wire when acted on by means of the fluid, such that the balloon 10 in the expanded state has the curved three-dimensional form of a banana.

FIG. 5 shows a schematic view of the balloon 10 in a variant of a balloon catheter according to the invention, wherein the balloon 10 has three wires 32, 33 and 34. In this case, the helical wire 34 is used as a frame for the balloon 10. In this variant of the invention, the rigidity of the balloon 10 is changed locally by the two wires 32 and 33. In this variant of the invention, the two wires 32 and 33 are connected fixedly to the windings of the helical wire 34. The connection is made in this case by threading the wires through. The length 132 and 133 between two adjacent windings of the helical wire 34 is key in this variant. In this variant of the invention the length 132 is much shorter than 133, whereby the rigidity of the balloon 10 along the wire 32 is increased considerably. The balloon 10 accordingly stretches less along the wire 32 when acted on by means of a fluid and adopts a curved three-dimensional form similar to a banana, similarly to the variant of the invention according to FIG. 4.

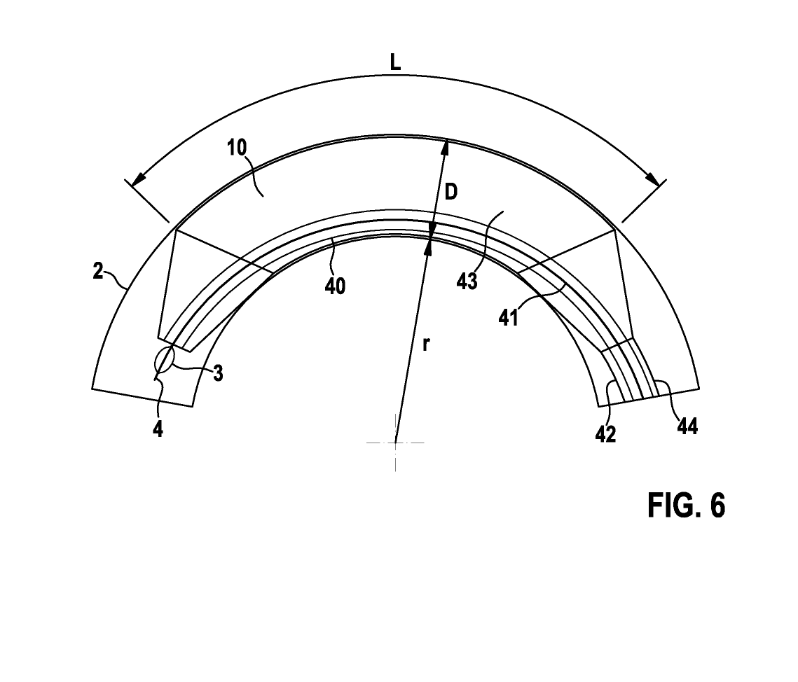

FIG. 6 shows a schematic variant of the invention, in which the length of the inner shaft 40 is shorter than the length of the balloon 10. The balloon catheter is illustrated in the bodily vessel 2 in this instance. The balloon catheter has an inner shaft 40 with an inner lumen 41 as well as an outer shaft 44 with an outer lumen 42. The outer lumen 42 is located between the inner shaft and outer shaft and is fluidically connected to the balloon interior 43. The balloon 10 is acted on by means of a fluid via the outer lumen 42 and is expanded. The guide wire 4 is located in the inner lumen 41. A tip 3 made of soft X-ray visible material is located at the distal end of the balloon catheter.

In this variant of the invention the length of the inner shaft 41 from the proximal end to the distal end of the balloon 10 is shorter than the length of the balloon. In this variant of the invention, a three-dimensional curved form of the balloon 10 is thus ensured in its expanded state. The inner shaft 40 follows the three-dimensional course of the vessel 2 directly. Since the length of the balloon is greater than the length of the inner shaft from the proximal end to the distal end of the balloon, it is ensured that the balloon adapts to the three-dimensional course of the bodily vessel 2 in the expanded state.

In the variant of the invention shown in FIG. 6, the length of the balloon 10 of diameter D from the proximal end to the distal end L*(1−r/r+D)) is greater than the length L of the inner shaft 40 from the proximal end of the balloon 10 to the distal end of the balloon 10, so as to follow a radius of curvature r of the bodily vessel 2. The length L of the balloon 10 is determined in this case without the conical end regions, and the diameter D is determined in the expanded sate.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A balloon catheter, comprising an expandable balloon for the treatment of stenoses in bodily vessels, characterized in that the balloon is formed in such a way that, in an expanded state, it adopts a form that deviates from the form of a cylinder, and in particular the balloon has a curved or wound three-dimensional form in the expanded state.

2. The balloon catheter as claimed in claim 1, characterized in that the balloon is composed from at least two, optionally three, individual segments, wherein at least one segment has an area that is not rectangular.

3. The balloon catheter as claimed in claim 2, characterized in that the individual segments are welded or glued together at their bordering edges.

4. The balloon catheter as claimed in claim 2, characterized in that the balloon, in the expanded state, has a form that comprises at least two cylindrical portions, wherein main axes of at least two cylindrical portions have an angle of more than 0° and less than 180°, optionally more than 0° and less than 90°.

5. The balloon catheter as claimed in claim 1, characterized in that at least one wire is suitably connected to the material of the balloon in such a way that the rigidity of the balloon in an unexpanded state differs in at least one portion along a first line over the balloon from the rigidity along a second line over the balloon.

6. The balloon catheter as claimed in claim 5, characterized in that the wire/the wires is/are welded or glued along a curve to the material of the balloon, preferably in the interior of the balloon, or is/are integrated into the balloon material.

7. The balloon catheter as claimed in claim 6, characterized in that the curve in the unexpanded state of the balloon is a straight line and optionally extends from a proximal end to a distal end of the balloon.

8. The balloon catheter as claimed in claim 5, characterized in that the curve is a helix, wherein, along a first straight axis over the balloon from a proximal end to a distal end, less balloon material is located between two adjacent points of intersection of a first curve and a helix than between the adjacent points of intersection of a second curve over the balloon from the proximal end to the distal end and the helix, wherein the first and second curve are straight lines in the unexpanded state of the balloon.

9. The balloon catheter as claimed in claim 8, characterized in that the helix and the first and second curve extend from the proximal end to the distal end of the balloon, wherein the first and second curve are optionally located on opposite sides of the balloon.

10. The balloon catheter as claimed in claim 1, characterized in that at least two wires are located inside the balloon and are interconnected in such a way that resilience of the balloon in an unexpanded state differs in at least one portion along a first line over the balloon from resilience along a second line over the balloon.

11. The balloon catheter as claimed in claim 10, characterized in that a helical wire is connected to a wire at least at two adjacent windings of the helix, wherein both wires preferably extend from a proximal end to a distal end of the balloon.

12. The balloon catheter as claimed in claim 10, characterized in that a helical wire is connected to a first wire at least at two adjacent windings of the helix and is connected to a second wire at least at two adjacent windings of the helix.

13. The balloon catheter as claimed in claim 12, characterized in that the length of the first wire between two adjacent windings of the helix differs from the length of the second wire between two adjacent windings of the helix, wherein both the helical wire and the first and second wire preferably extend from the proximal end to the distal end of the balloon.

14. The balloon catheter as claimed in claim 1, which has in the distal region an inner shaft with an inner lumen and an outer second lumen, wherein the second lumen has a fluidic connection to the balloon interior in such a way that the balloon can be expanded by a fluid in the second lumen, characterized in that the length of the balloon from a proximal end to a distal end is greater than the length of the inner shaft from the proximal end of the balloon to the distal end of the balloon.

15. The balloon catheter as claimed in claim 14, characterized in that the length of the balloon of diameter d from the proximal end to the distal end L*(131 r/(r+d)) is greater than the length L of the inner shaft from the proximal end of the balloon to the distal end of the balloon so as to follow a radius of curvature r of the bodily vessel.

16. The balloon catheter as claimed in claim 14, characterized in that the balloon catheter has means, with which the size of the difference in length between the balloon and the inner shaft can be set variably, and in particular the distal end of the balloon is pressure tight and movable over the inner shaft.
